(19) United States
(12) Patent Application Publication
(10) Pub. No.: US 2008/0287965 A1
(43) Pub. Date: Nov. 20, 2008

(54) RADIOPAQUE BAND LIGATOR

(76) Inventor: Richard Ducharme, Winston-Salem, NC (US)

Correspondence Address: BRINKS HOFER GILSON & LIONE/CHICAGO/COOK, PO BOX 10395, CHICAGO, IL, 60610, US

(21) Appl. No.: 12/121,577

(22) Filed: May 15, 2008

Related U.S. Application Data
(60) Provisional application No. 60/930,759, filed on May 17, 2007.

(51) Int. Cl. A61B 17/22; A61B 6/00

(52) U.S. Cl. 606/140; 600/431

(57) ABSTRACT

The technology described herein relates to a system for capturing tissue with one or more ligating bands comprising radiopaque material that are removably disposed on a ligating barrel and methods of marking a bodily tissue for treatment using the ligating systems comprising one or more ligating bands comprising radiopaque materials. Also disclosed are methods of non-invasively evaluating the rate of amelioration of a bodily organ comprising capturing the tissue with one or more ligating bands comprising radiopaque materials, observing the location of the bands, and correlating the location of the one or more ligating bands and the rate of amelioration of the bodily organ.

### CROSS-REFERENCE TO RELATED APPLICATION

This application claims the benefit of U.S. Provisional Application No. 60/930,759, filed May 17, 2007, which is incorporated by reference in its entirety.

### TECHNICAL FIELD

This disclosure relates to systems and methods for marking a bodily tissue so that the tissue may be non-invasively monitored.

### BACKGROUND

The treatment of tissue encompasses a variety of techniques such as electrocauterization, heat therapy, resection (removal of tissue), and sclerotherapy (the injection of medicine into bodily tissue). These treatment techniques usually involve the passing of medical instruments through the operating channel of the endoscope. The endoscope permits minimally invasive access, as well as visualization and suction aids.

Another technique that frequently utilizes the operating channel of the endoscope is ligation, which involves applying a band or ligature around a vessel or portion of tissue, thereby cutting off blood or fluid flow and causing the tissue to necrose and separate from adjacent healthy tissue. Ligation is widely used to treat a number of medical tissue conditions, including, but not limited to, hemorrhoids, polyps, ballooning varices, and other types of lesions, including those that are cancerous. Typically, ligators are also used with a suction or vacuum means to draw the tissue into the distal end, whereby the band is deployed over the base of the diseased tissue to cut off blood flow. The ligating device is typically activated by retracting a line (string, wire, or cable) that is attached to the ligator at the distal end of an endoscope and is threaded through the operating channel of the endoscope to the proximal end of the instrument. The ligator can be activated by mechanically pulling the activating line by means of a hand-operated reel or trigger, or by a motor drive mechanism. Various other ligating devices use cooperating inner and outer members that slide the individual bands by pushing or pulling them from the end of the inner or outer member, the bands being preloaded onto the inner or outer member prior to deployment.

Band ligators are also used to mark tissue and treat both esophageal varices and hemorrhoids. However, they cannot be observed non-invasively. Therefore, there is a need for ligating bands that would allow a physician to identify by x-ray or under fluoroscopy the location of these bands entirely non-invasively. Another advantage of this method compared to other types of tissue devices such as hemostasis clips is that the tissue can be marked multiple times without having to remove a ligating device. Such a technique may be useful in evaluating the healing process non-invasively.

### BRIEF SUMMARY

Herein provided is a system used to mark a bodily tissue. The system comprises an activating component, a ligating barrel operably connected to the activating component, and one or more ligating bands comprising radiopaque material that are removably disposed on the ligating barrel.

Also provided is a method of marking a bodily tissue for treatment. The method comprises positioning a ligating barrel over a bodily tissue. The bodily tissue is drawn into the ligating barrel and marked by deploying one or more ligating bands around the bodily tissue. The bodily tissue is then released from the ligating barrel. The one or more ligating bands comprise a radiopaque material. The method further comprises the step of observing the one or more ligating bands under fluoroscopy.

In another aspect of the present invention, a method of non-invasively evaluating the rate of amelioration of a bodily tissue is provided. The method comprises capturing the bodily tissue, in some embodiments, by using a ligating system, and deploying one or more ligating bands around the bodily tissue. The location of the one or more ligating bands is observed relative to a bodily organ. An observer can correlate the location of the one or more ligating bands and the rate of amelioration of the bodily organ.

### BRIEF DESCRIPTION OF THE DRAWINGS

FIG. 1 is a longitudinal fragmented view of an endoscope with a ligating system.

FIG. 2 is a top plan view of the activating mechanism of the ligating mechanism.

FIG. 3A illustrates a cutaway view of the stomach with the distal end of an endoscope positioned over a gastric varix.

FIG. 3B is a close up view of the ligating barrel with radiopaque bands placed over the varix.

FIG. 4 is a close up view of the varix being pulled into the ligating barrel by suction.

FIG. 5 is a depiction of the varix marked with a radiopaque band.

FIG. 6 illustrates the marked tissue and radiopaque bands passing through the gastrointestinal tract.

### DETAILED DESCRIPTION OF THE DRAWINGS AND THE PRESENTLY PREFERRED EMBODIMENTS

The present invention provides a ligating system used to mark a bodily tissue. The system is useful in general band ligation, treatment of esophageal varices, and the treatment of normal or abnormal tissue in the gastrointestinal, respiratory, urinary, or alimentary tracts or even the female reproductive system. Abnormal tissue is considered tissue that is cancerous or precancerous.

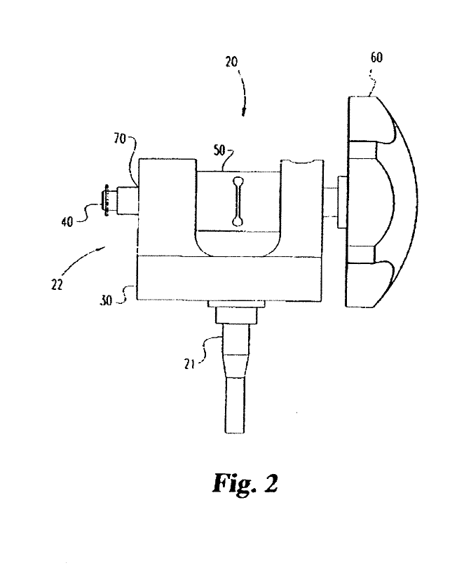

The system comprises an activating component used in deploying the ligating bands. In some embodiments that may be preferred, the system comprises an activating component adapted to be connected to the proximal end of an endoscope operating channel. FIG. 2 is a top plan view of an activating mechanism 20 for some embodiments of the ligating system disclosed. The activating mechanism 20 comprises a mounting component 21 and an activating component 22. The activating component 22 includes a base 30, a drive pin 40, a spool 50, a knob 60, and a roller clutch 70. The mounting component 21 attaches to the activating component 22 and is used to mount the activating mechanism 20 to the endoscope 10 at a proximal opening 18. Although an activating component 22 having a knob 60 and roller clutch 70 is shown, other embodiments of the invention can utilize different activating components.

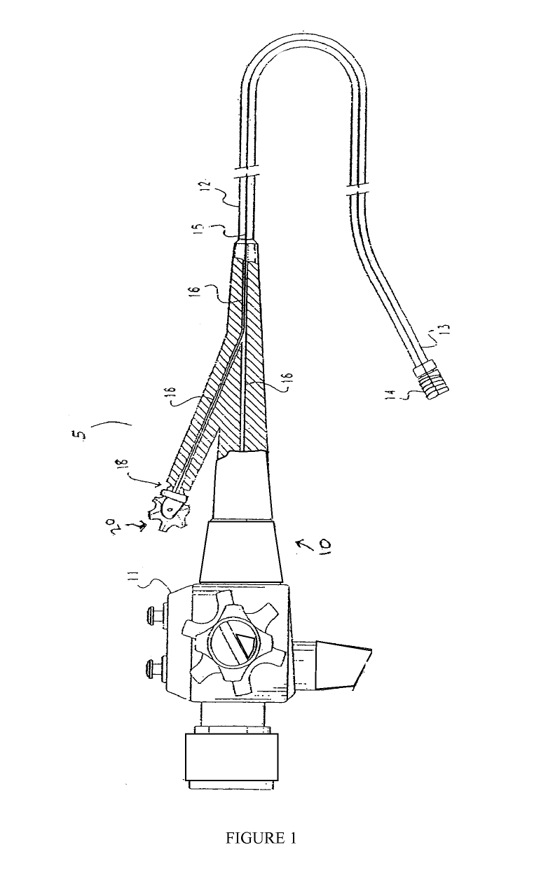

The ligating system also comprises a ligating barrel 14 operably connected to the activating component 22. The ligating barrel 14 is adapted to be disposed on a distal end of an endoscope 10 in some embodiments. As shown in FIG. 1, the ligating system 5 has a ligating barrel 14 attached to the distal end of an endoscope 10. The ligating barrel 14 is generally shown in FIG. 1, but a more detailed description of the ligating barrel 14 can be found in U.S. Pat. No. 5,624,453, which disclosure is hereby incorporated by reference. The endoscope 10 is a conventional endoscope with an operating control portion 11, a flexible section 12, and a distal end portion 13. Although an endoscope is used in some preferred embodiments, there are embodiments that may be preferred where the radiopaque bands are used in conjunction with hemorrhoid band ligators and anoscope systems.

In some embodiments, the system further comprises an activation line 15 operably connected between the activating component 22 and the ligating barrel 14. As shown in FIG. 1, the ligating barrel 14 is located at the distal end portion 13 of the endoscope 10 and includes an activation line 15. The activation line 15 is coupled to the activating component 22 and the ligating bands. Using an activation line 15 or lines to release one or more ligating bands is described generally in U.S. Pat. No. 6,730,101, the entire contents of which are hereby incorporated herein by reference. In some embodiments, one or more ligating bands are coupled to the activating component 22 via an activation line 15 or lines. Operation of the activating component 22 releases the one or more ligating bands from the ligating barrel 14 to mark a bodily tissue.

The endoscope 10 also includes an operating channel 16 which extends through the endoscope 10 from the ligating barrel 14 to both the operating control portion 11 and to the proximal opening 18. The activation line 15 is threaded from the ligating barrel 14 through the operating channel 16 and exits through the proximal opening 18. The barrel 14 is preferably of a hard plastic clear polycarbonate for maximum durability and visibility.

The system comprises one or more ligating bands 45 comprising radiopaque material. The bands 45 are removably disposed on the ligating barrel 14. The radiopaque bands 45 are generally elastic as they recover their size and shape after deformation. The ligating system may comprise as many as six (or more) deployable ligating bands 45 disposed on the ligating barrel. An operator is therefore capable of banding or marking as many as six (or more) separate tissue sections (i.e., pseudo-polyps) without withdrawing the endoscope 10 from the patient to reload the ligating system with additional ligating bands 45.

The ligating bands 45 of the present invention comprise latex, neoprene, silicone, metal, or any substance exhibiting radiopaque properties suitable for band ligation. The ligating bands are made with radiopaque materials that comprise barium sulfate, tungsten, bismuth, or other material exhibiting radiopaque qualities. Two or more materials are used in combination in some embodiments of the present invention. In some embodiments, below 12% by weight of radiopaque material can be blended with the ligating bands. In embodiments that may be preferred, below 10% by weight of radiopaque material can be used or below 8% in others. Although some of these radiopaque materials are in powder form, there are embodiments wherein the radiopaque material is injectable, for instance, radiopaque latex. In some other embodiments, the radiopaque material can be added to the band by spray coating or electrospinning. In embodiments where the ligating bands comprise metal, parylene is coated over the band to help enclose the metal.

The systems of the present invention are useful in methods of marking a bodily tissue for treatment. In some embodiments, the method comprises providing a ligating system as described above. The ligating barrel 14 is placed on the distal end of the endoscope 10 and the activating component 22 is connected to the proximal opening of the operating channel 16. The activation line 15 is disposed through the operating channel 16 to operably connect the activation line 15 between the ligating barrel 14 and the activating component 22.

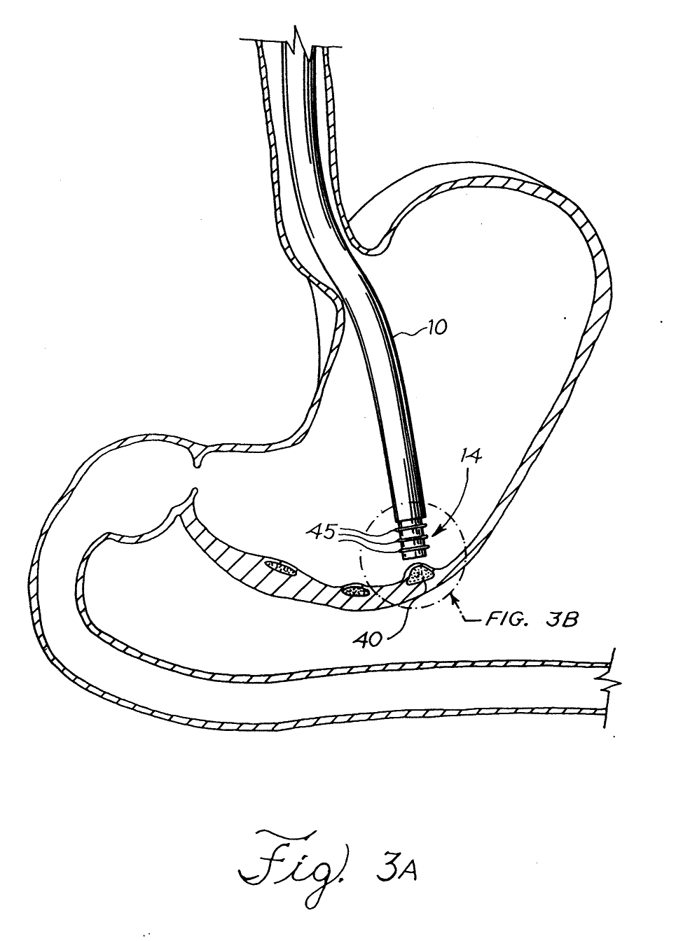

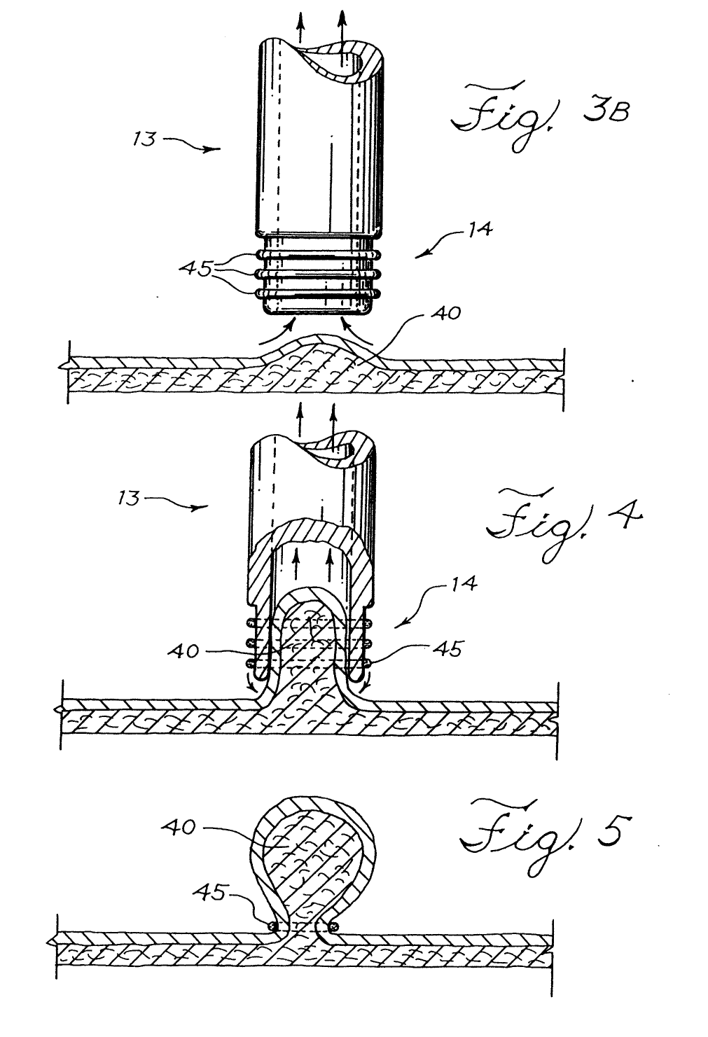

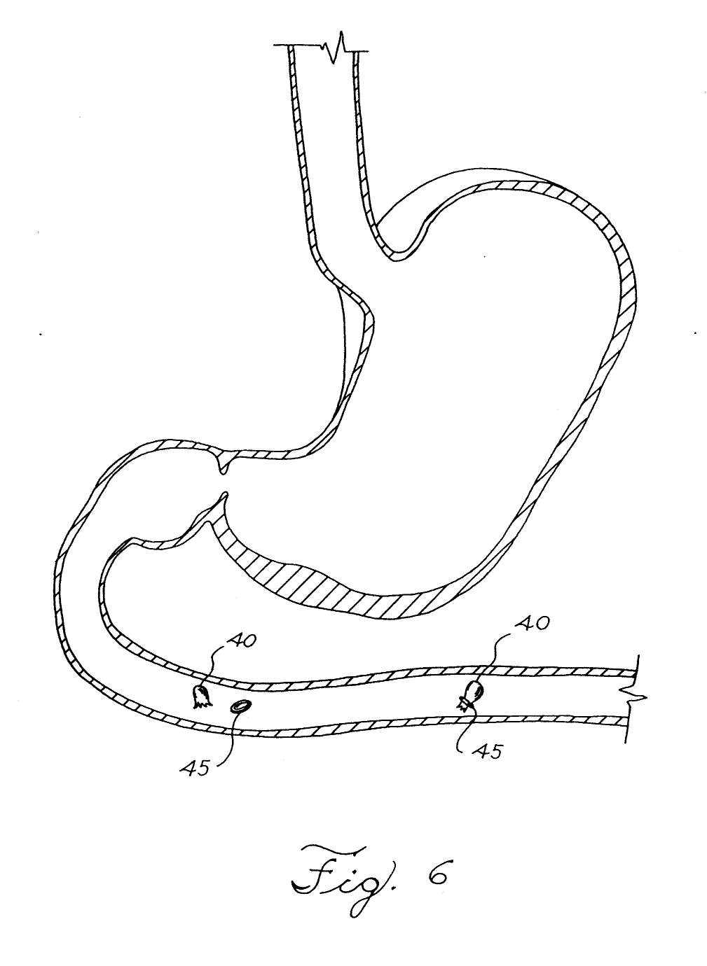

The methods of the present invention are therefore useful in the treatment of gastric varices. As shown in FIG. 3A, the ligating barrel 14 is positioned over a gastric varix 40 to be marked. The varix 40 is drawn into the ligating barrel 14 by suction or other means known in the art as shown in the insert, FIG. 3B, and in FIG. 4. One or more ligating bands 45 are released around the varix 40 by actuation of the activating component 22. In FIG. 5, the varix 40 is released from the ligating barrel 14 marked with a radiopaque ligating band 45. Other bodily tissues that may be marked using these methods include, but are not limited to, a polyp, varix, cancer, or other lesion.

Releasing the bands 45 around the varix 40 marks the varix 40 with radiopacity so that it may be tracked at a later time. The activating component 22 pulls the activation line 15 proximally to release the one or more ligating bands 40 from the ligating barrel 14 in some embodiments. There are also embodiments where the ligating system has other means for releasing the bands. Ligating systems with dual barrels, pushrods, pullstrings, or any other ligating system suitable for deploying ligating bands may also be used.

After the varix 40 is marked with a radiopaque band 45, the varix 40 can be observed using fluoroscopy, X-ray, magnetic resonance imaging, or other means suitable for monitoring radiopaque objects. Accordingly, the present invention provides a method of non-invasively evaluating the rate of amelioration of a bodily organ. The method comprises marking a bodily tissue in the bodily organ with one or more radiopaque ligating bands, observing the location of the one or more bands relative to the bodily organ, and correlating that location with the rate of amelioration of the bodily organ. The location of the radiopaque bands can be observed at predetermined intervals of time or randomly throughout a patient's course of treatment.

The radiopaque band may cause necrosis in a marked tissue. Once the tissue has necrosed, the tissue along with the band passes through the body as depicted in FIG. 6. As such, the methods of the present invention allow monitoring to determine if the marked tissue has necrosed and monitoring to determine the location of the band as it passes through the body. In this example, the position of the band 45 can be monitored after the varix 40 necroses and as they both passes through the bodily system. In some instances, the band 45 may become separated from the varix 40. The position of the band 45 is still relevant as it is still a sign that the varix 40 has necrossed. In some methods, the marked tissue is observed for later biopsy, possibly by a different surgeon. In other methods, a hemorrhoid is marked for later chemical destruction. Although in some of the methods mentioned above capture the bodily tissue by a ligating barrel coupled to an endoscope, an anoscope may also be used in other embodiments.

It will, of course, be well understood from the discussion above that other known ligating barrel designs, activation mechanisms, endoscope systems, etc. could be used within the scope of the invention. It is therefore intended that the foregoing detailed description be regarded as illustrative rather than limiting, and that it be understood that it is the following claims, including all equivalents, that are intended to define the spirit and scope of this invention.

1. A ligating system used to mark a bodily tissue, the system comprising:
an activating component;
a ligating barrel operably connected to the activating component; and
one or more ligating bands comprising radiopaque material, the one or more ligating bands being removably disposed on the ligating barrel.

2. The system of claim 1 wherein the radiopaque material comprises barium sulfate, tungsten, bismuth, or injectable latex.

3. The system of claim 1 further comprising an activation line operably connected between the activating component and the ligating barrel.

4. The system of claim 3 wherein the one or more ligating bands are coupled to the activating component via the activation line, wherein operation of the activating component releases the one or more ligating bands from the ligating barrel.

5. A method of marking a bodily tissue for treatment, the method comprising:
positioning a ligating barrel over a bodily tissue;
drawing the bodily tissue into the ligating barrel; and
capturing the bodily tissue by releasing one or more ligating bands comprising radiopaque material around the bodily tissue, wherein the capturing step marks the bodily tissue for observation.

6. The method of claim 5 wherein the bodily tissue is a hemorrhoid, polyp, varix, cancer, or other lesion.

7. The method of claim 5 wherein the radiopaque material comprises barium sulfate, tungsten, or bismuth.

8. The method of claim 5 wherein the bodily tissue is drawn into the ligating barrel by suction.

9. The method of claim 5 wherein the ligating barrel is operably connected to an activating component and is adapted to be disposed on the distal end of an endoscope.

10. The method of claim 5 wherein the ligating system further comprises an activating component being adapted to be connected to a proximal end of an endoscope operating channel.

11. The method of claim 5 wherein the system further comprises an activation line operably connected between an activating component and the ligating barrel.

12. The method of claim 5 further comprising disposing the ligating barrel on the distal end of the endoscope, connecting an activating component to the proximal opening of an operating channel, disposing the activation line through the operating channel, and operably connecting the activation line between the ligating barrel and the activating component.

13. The method of claim 11 wherein operation of the activating component pulls the activation line proximally to deploy the one or more ligating bands from the ligating barrel.

14. The method of claim 5 further comprising observing the location of the one or more ligating bands.

15. The method of claim 14 wherein the location is observed by fluoroscopy or magnetic resonance imaging.

16. A method of non-invasively evaluating the rate of amelioration of a bodily organ, the method comprising:
capturing a bodily tissue in the bodily organ with one or more ligating bands, wherein the capturing step marks the bodily tissue for observation, the one or more ligating bands comprising radiopaque material;
observing the location of the one or more ligating bands relative to the bodily organ; and
correlating the location of the one or more ligating bands and the rate of amelioration of the bodily organ.

17. The method of claim 16 wherein the bodily tissue comprises a hemorrhoid, polyp, varix, cancer, or other lesion.

18. The method of claim 16 wherein the radiopaque material comprises barium sulfate, tungsten, bismuth, or injectable latex.

19. The method of claim 16 wherein the location is observed by X-ray, fluoroscopy, or magnetic resonance imaging.

20. The method of claim 16 wherein the bodily tissue is captured by a ligating barrel coupled to an endoscope or by an anoscope.
